(19) United States
(12) Patent Application Publication
(10) Pub. No.: US 2024/0314899 A1
(43) Pub. Date: Sep. 19, 2024

(54) IPL STERILIZATION DEVICE

(71) Applicant: IUCF-HYU (INDUSTRY-UNIVERSITY COOPERATION FOUNDATION HANYANG UNIVERSITY), Seoul (KR)

(72) Inventors: Young Beom KIM, Seoul (KR); Jongheon KIM, Seoul (KR); Sungkeun PARK, Seoul (KR); Hansam LEE, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/576,201

(22) PCT Filed: Jul. 5, 2022
(86) PCT No.: PCT/KR2022/009649

§ 371 Date: Jan. 3, 2024

(30) Foreign Application Priority Data
Jul. 5, 2021 (KR) 10-2021-0087832

(51) Int. Cl. H05B 41/36; A61L 2/08; H01J 61/02; H02J 7/34

(52) U.S. Cl. CPC H05B 41/36; A61L 2/084; H01J 61/025; H02J 7/345; A61L 2202/11; A61L 2202/14; H02J 2207/50

(57) ABSTRACT

According to an embodiment, an IPL sterilization device includes a lamp configured to output light including a visible light region to sterilize a region including a surface of an object; a capacitor configured to transmit a voltage charged to the lamp; and a controller configured to control the capacitor, wherein the controller is configured to: control the lamp to be driven in an outputable state or an unoutputable state, change the lamp to the unoutputable state when the object satisfies an object overheat condition in the outputable state, and change the lamp to the outputable state when the object unsatifies the object overheat condition after the lamp satisfies the object overheat condition and changes the lamp to the unoutputable state, wherein the outputable state is a state in which the lamp is capable of outputting light by applying a driving pulse to the lamp by the capacitor, and the unoutputable state is a state in which the lamp is not capable of outputting light because the capacitor does not output a driving voltage to the lamp.

### TECHNICAL FIELD

An embodiment provides an IPL sterilizer.

### BACKGROUND ART

Recently, as the virus problem has been emerged as a serious problem in the world, interest in sterilization has increased. The conventional sterilizer device is mostly using an ultraviolet light source, but in the case of a sterilizer device that uses ultraviolet light, it takes a lot of time to sterilize, so sterilization is rarely achieved by short-term ultraviolet irradiation, and the ultraviolet light used is harmful to the human body.

In order to compensate for the disadvantages of the sterilizer device that uses ultraviolet light, an IPL (Intense Pulsed Light) sterilizer device has been developed as a sterilizer device. The IPL sterilization device is a device that irradiates light of a short pulse type with a strong intensity to a subject to sterilize the subject, and the subject absorbs the energy of the light irradiated from the IPL sterilization device, so that the surface temperature rapidly rises, and the microorganisms and viruses on the surface of the subject are killed, so that the subject is sterilized. However, the IPL sterilization device is not popular than the ultraviolet sterilizer device.

Therefore, there is a need to develop a technique for a sterilizer device that uses IPL to sterilize the subject.

### DISCLOSURE

### Technical Problem

An embodiment provides an IPL sterilization device that sterilizes a subject using the IPL technology.

An embodiment provides an IPL sterilization device that controls an operation state based on an object overheat condition of an object.

The technical problems that are not mentioned herein are not limited to the above technical problems, and the technical problems that are not mentioned herein can be clearly understood by those skilled in the art from the present specification and the accompanying drawings.

### Technical Solution

According to an embodiment, an IPL sterilization device includes a lamp configured to output light including a visible light region to sterilize a region including a surface of an object; a capacitor configured to transmit a voltage charged to the lamp; and a controller configured to control the capacitor, wherein the controller is configured to: control the lamp to be driven in an outputable state or an unoutputable state, change the lamp to the unoutputable state when the object satisfies an object overheat condition in the outputable state, and change the lamp to the outputable state when the object unsatifies the object overheat condition after the lamp satisfies the object overheat condition and changes the lamp to the unoutputable state, wherein the outputable state is a state in which the lamp is capable of outputting light by applying a driving pulse to the lamp by the capacitor, and the unoutputable state is a state in which the lamp is not capable of outputting light because the capacitor does not output a driving voltage to the lamp.

The means for solving the technical problems of the present invention are not limited to the above-described means, and the means for solving the technical problems not mentioned may be clearly understood by those skilled in the art to which the present invention pertains from this specification and the accompanying drawings.

### Advantageous Effects

The IPL sterilization device according to the embodiment may efficiently sterilize the subject in a short time harmlessly to the human body using the IPL technology.

The IPL sterilization device according to the embodiment may monitor the object overheat condition of the object, and when the object is in the overheat condition, the IPL sterilization device may be controlled to be in an unoutputable state to prevent the object from being damaged due to the overheating of the object, and may perform a safer sterilization operation.

The effects of the present application are not limited to the above-described effects, and the effects that are not mentioned may be clearly understood by those skilled in the art from the present specification and the accompanying drawings.

### DESCRIPTION OF DRAWINGS

FIGS. 1. to 3 are diagrams for explaining an IPL sterilization device according to an embodiment.

FIG. 4 is a circuit diagram illustrating the circuit unit 140 of the IPL sterilization device according to the first embodiment.

FIG. 5 is a circuit diagram illustrating when the circuit unit of the IPL sterilization device is in a charging state according to the first embodiment.

FIG. 6 is a diagram illustrating the capacitor voltage and the light output from the lamp when the IPL sterilization device is in a charging state according to the first embodiment.

FIG. 7 is a circuit diagram illustrating when the circuit unit of the IPL sterilization device is in a light-emitting state according to the first embodiment.

FIG. 8 is a diagram illustrating a capacitor voltage and light output from a lamp when the IPL sterilization device is in a light-emitting state according to the first embodiment.

FIG. 9 is a diagram illustrating a sensor unit according to the first embodiment.

FIG. 10 is a flowchart illustrating the operation of the IPL sterilization device according to the first embodiment, and

FIG. 11 is a waveform diagram related to the operation of the IPL sterilization device according to the first embodiment.

FIG. 12 is a diagram illustrating the object overheat condition leaving in the unoutputable state of the IPL sterilization device according to the first embodiment.

FIG. 13 is a diagram for explaining the object overheat condition determination according to an illuminance sensor in an IPL sterilization device according to the first embodiment.

FIG. 14 is a flowchart illustrating the driving of the IPL sterilization device according to the second embodiment, and

FIG. 15A and FIG. 15B is a waveform diagram illustrating the standby condition driving according to the second embodiment.

FIG. 16 is a diagram illustrating a circuit unit of an IPL sterilization device according to the third embodiment.

FIG. 17 is a circuit diagram illustrating a case where the circuit unit of the IPL sterilization device is in a charging state according to the third embodiment.

FIG. 18 is a diagram illustrating a capacitor voltage and light output from a lamp when the IPL sterilization device is in a charging state according to the third embodiment.

FIG. 19 is a circuit diagram illustrating a case where the circuit unit of the IPL sterilization device is in a light emission state according to the third embodiment.

FIG. 20 is a diagram illustrating a capacitor voltage and light output from a lamp when the IPL sterilization device is in a light emission state according to the third embodiment.

### BEST MODE

The above-described objects, features, and advantages of the present invention will become more apparent from the following detailed description related to the accompanying drawings. However, the present invention may be variously modified and have various embodiments, and specific embodiments are illustrated in the drawings and will be described in detail below.

In the drawings, the thickness of layers and regions is exaggerated to ensure clarity, and an element or layer referred to as another element or layer “on” or “on” includes both the case where another layer or another element is interposed in the middle as well as the immediately above the other element or layer. Throughout the specification, the same reference numerals in principle indicate the same elements. In addition, functions within the same scope of the drawings of each embodiment will be described using the same reference numerals.

Detailed descriptions of known functions or constructions related to the present invention will be omitted when it is determined that they may unnecessarily obscure the subject matter of the present invention. In addition, the numbers (e.g., first, second, and the like) used in the description process of this specification are only identification symbols for distinguishing one element from another element.

In addition, the suffix words “module” and “unit” for the elements used in the following description are given or mixed only in consideration of the ease of writing the specification, and do not have a distinguishing meaning or role by itself.

An IPL sterilization device may be configured to include a lamp configured to output light including a visible light region to sterilize a region including a surface of an object; a capacitor configured to transmit a voltage charged to the lamp; and a controller configured to control the capacitor, wherein the controller is configured to: control the lamp to be driven in an outputable state or an unoutputable state, change the lamp to the unoutputable state when the object satisfies an object overheat condition in the outputable state, and change the lamp to the outputable state when the object unsatifies the object overheat condition after the lamp satisfies the object overheat condition and changes the lamp to the unoutputable state, wherein the outputable state is a state in which the lamp is capable of outputting light by applying a driving pulse to the lamp by the capacitor, and the unoutputable state is a state in which the lamp is not capable of outputting light because the capacitor does not output a driving voltage to the lamp.

The controller may be configured to determine the object overheat condition when the temperature measured by a temperature sensor is higher than a predetermined temperature.

The controller may be configured to determine the object overheat condition when the IPL sterilization device does not move during a predetermined period of time.

The controller may be configured to determine the object overheat condition when a driving pulse is output a predetermined number of times while a sensing value output by one of a motion sensor, an illuminance sensor, or a contact sensor is within a certain range.

The controller may be configured to determine the object overheat condition when a variation in a sensing value detected by one of a motion sensor, an illuminance sensor, or a touch sensor is within a predetermined range during a predetermined period of time.

The controller may be configured to change the lamp to the outputable state when a predetermined period of time has elapsed while the object overheat condition is not satisfied after changing to the unoutputable state.

An IPL sterilization device may be configured to include an illuminance sensor configured to measure the illuminance of the object, and the controller is configured to: determine the object overheat condition when the sensing value measured by the illuminance sensor is maintained for a first period of time with a first value, and determine the object overheat condition of the object when the sensing value measured by the illuminance sensor is maintained for a second period of time with a second value, wherein the first value is a larger value than the second value, and the first period is longer than the second period.

The illuminance sensor may be configured to measure a color or an illuminance of the object in a section where the lamp emits.

The illuminance sensor may be configured to measure a color or illuminance of the object in a section other than the section where the lamp emits.

The controller may be configured to control the lamp to be driven in a standby state when the standby condition, wherein the standby condition is a condition that has a temperature lower than a temperature of the object overheat condition.

The controller may be configured to control a pulse having a different pulse width from the driving pulse to be applied to the lamp from the capacitor when the lamp is in the standby condition.

The pulse width applied to the lamp in the standby condition may be smaller than the driving pulse applied to the lamp in the outputable state.

An IPL sterilization device may include a light shielding structure for blocking light output from the lamp from being incident to the illuminance sensor.

### MODE FOR INVENTION

Hereinafter, an IPL apparatus according to an embodiment will be described with reference to the drawings.

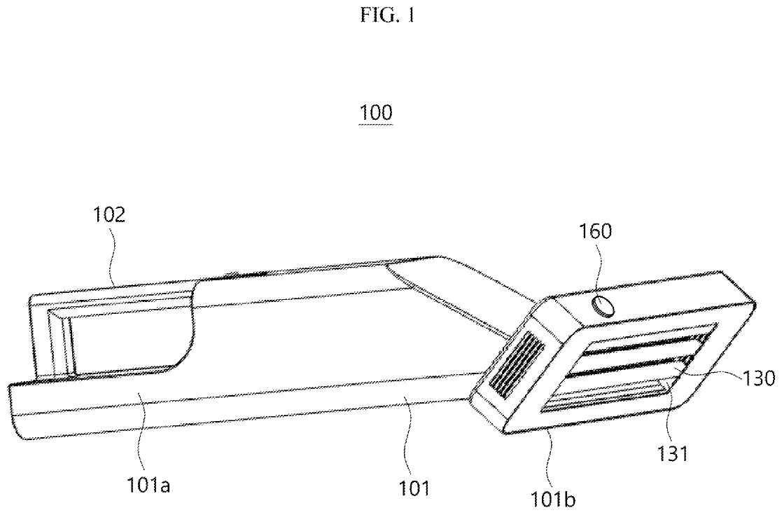

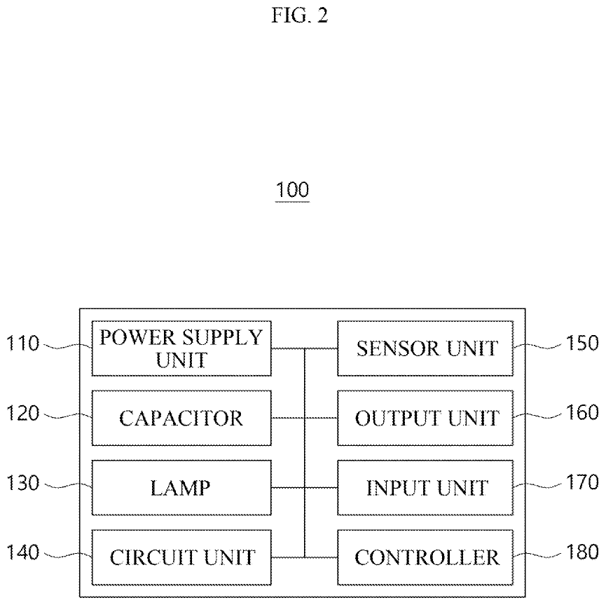

FIGS. 1 to 3 are diagrams for explaining an IPL sterilization device according to an embodiment.

Referring to FIGS. 1 and 2, the IPL sterilization device 100 may include a body 101, a handle 102, a power supply 110, a capacitor 120, a lamp 130, a circuit unit 140, a sensor unit 150, an output unit 160, an input unit 170, and a controller 180. The arrangement of the components is not limited to the illustration of FIG. 1, and the components may be disposed at any position to execute functions to be applied to the IPL sterilization device 100.

The body 101 may be implemented in various shapes. For example, the body 101 may be formed in a t-shape as a whole. Of course, the body 101 may be implemented in various shapes such as an i-shape or a cube-shape.

The handle 102 may be disposed on the body 101 so that the user may house when the IPL sterilization device 100 is used. For example, the body 101 may include a first body portion 101a in which the handle 102 is installed and a second body portion 101b in which the lamp 130 is installed for sterilization. As an example, the user may pick the IPL sterilization device 100 through the handle 102, place the second body portion 101b in which the lamp 130 is installed close to the area or the object to be sterilized, and output pulse light through the lamp 130.

The components of the IPL sterilization device 100 may be disposed on the body 101. For example, the lamp 130, the output unit 160, and the handle 102 may be installed on the body 101. In addition, components such as the power supply unit 110, the capacitor 120, the circuit unit 140, the sensor unit 150, and the controller 180, which are not shown in the drawings, may also be installed inside or outside the body 101. Of course, each component can be installed inside or outside the handle 102, etc., but is not limited to the above description.

The IPL sterilization device 100 may include a wheel 191 in one area of the second body portion 101b to maintain contact with the sterilized body and facilitate movement. For example, the wheel 191 may be disposed under the second body portion 101b or in one region of the light guide unit 131.

Referring to FIG. 3(a), which is a schematic cross-sectional view of the IPL sterilization device 100, the wheel 191 disposed under the second body portion 101b may contact the area to be sterilized and rotate by the force of the user. For example, the wheel 191 may be formed in a structure in which light output from the lamp 130 does not leak outside. Specifically, the wheel 191 may be formed in a structure that protrudes more than a certain portion of the light guide unit 131 and surrounds it. Here, a plurality of wheels 191 may be used to surround the light guide unit 131 or a wheel having a wide area to surround the light guide unit 131.

When the pulse light output by outputting a strong intensity pulse light for sterilization leaks outside, the IPL sterilization device 100 may lose the user's eye when the IPL sterilization device 100 is used for sterilization, and the strong intensity light may be instantaneously applied to the user's eye and may be harmful to eye health. The IPL sterilization device 100 may include a light blocking unit 190 disposed in one area of the body 101 to reduce the leakage of the pulse light output from the lamp 130 to the outside. For example, the light blocking unit 190 may be disposed to surround the lower portion of the body 101. In addition, the light blocking unit 190 may be integrally formed with the light guide unit 131 to be described later.

Alternatively, a wheel 191 may be installed at one end of the light blocking unit 190 to maintain contact with the sterilized body and facilitate movement. For example, the light blocking unit 190 may be formed of a structure capable of accommodating the wheel 191 (for example, a groove is recessed to have a size equal to the wheel 191), and the wheel 191 having a size matched to the secured space may be installed to reduce light leakage to the outside. In other words, light output from the lamp 130 may be prevented from leaking to the outside by the light blocking unit 190 and the wheel 191. Referring to FIG. 3(b), which is a schematic cross-sectional view of the IPL sterilization device 100, the wheel 191 disposed at one end of the light blocking unit 190 may contact an area to be sterilized and rotate by a user's force.

The body 101 is not limited to the above-described description, such as additionally provided with a bumper (not shown) that buffers the impact.

The power supply 110 may provide current to drive at least one component of the IPL sterilization device 100. For example, the power supply 110 may provide current to drive the capacitor 120, the lamp 130, the circuit unit 140, the sensor unit 150, the output unit 160, the input unit 170, and the controller 180.

The power supply 110 may obtain external power or internal power under the control of the controller 180 and supply power to each of the components included in the IPL sterilization device 100. For example, the power supply 110 may include a battery, and the battery may be an internal type of battery or a replaceable type of battery.

For another example, the IPL sterilization device 100 may be implemented in a form of providing current to operate by connecting a power plug independently provided on one side of the IPL sterilization device 100 to a receptacle.

The power supply unit 110 may use a conventional power supply unit, and thus detailed descriptions will be omitted.

The capacitor 120 may accumulate electrical energy charged by the capacitor charger 143. The capacitor 120 may be a common capacitor generally used in a circuit, and detailed descriptions are omitted.

The lamp 130 may output pulsed light. For example, the lamp 130 may sterilize a subject in a region to be sterilized by outputting a strong pulse light in a short pulse format, that is, an intense pulsed light (IPL).

The lamp 130 may emit light of a complex wavelength. For example, the lamp 130 may obtain electrical energy and output light having a wavelength including a visible light band. For example, the lamp 130 may emit a complex wavelength of 400-1200 nm, not a single wavelength of light. Various filters may be used in the lamp 130 to irradiate rays of a desired wavelength band.

The lamp 130 may be disposed in one region of the body 101 to irradiate pulsed light of a wavelength band in the visible light region of 400 to 1200 nm to the subject to be sterilized. For example, the lamp 130 may be provided as a flash lamp such as a xenon lamp, and may emit light using supplied electrical energy. Of course, the lamp 130 may be composed of incandescent lamps, xenon lamps, laser diodes, leds, or a combination of two or more of these and others, but is not limited thereto.

A light guide unit 131 for guiding pulse light output from the lamp 130 may be disposed around the lamp 130. For example, the light guide unit 131 may be disposed in one area around the lamp 130, and guide pulsed light such that the pulsed light output from the lamp 130 is irradiated to at least some areas of the organism to be sterilized. To this end, the light guide unit 131 may extend a predetermined length around the lamp 130 to protrude a part thereof.

The light guide unit 131 may be implemented in various shapes. For example, the light guide unit 131 may have a concave shape toward the lamp 130. For example, the light guide unit 131 may be formed in a u-shaped structure as a whole so that the lamp 130 may be provided in an internal space to guide light output from the lamp 130. Here, one end of the light guide unit 131 may protrude more than the lamp 130 toward the sterilized area.

In addition, a reflective surface may be formed on the light guide unit 131. For example, the reflection surface may be installed at the rear of the lamp 130 to reflect the light output from the lamp 130 to reach the object to be sterilized. For another example, the reflective surface may be provided in a shape in which light output from the lamp 130 is collected toward the organism to be sterilized. The light guide unit 131 may easily reach the light output from the lamp 130 to the organism to be sterilized so that the organism may be effectively sterilized.

The circuit unit 140 may control the pulsed light output through the lamp 130 in various ways. The detailed configuration and operation of the circuit unit 140 will be described below.

The sensor unit 150 may include various sensors for detecting the ambient environment of the IPL sterilization device 100 or the state of the IPL sterilization device 100. The detailed configuration and operation of the sensor unit 150 will be described below.

The output unit 160 may output information related to the state of the IPL sterilization device 100. For example, the output unit 160 may include an indicator that indicates information related to the state of the IPL sterilization device 100 to the outside in various ways. For example, the indicator may be implemented as a LED lamp, a speaker, a motor, a haptic device, a vibrator, a signal output circuit, or the like. The indicator may visually or audibly output a start notification, a termination notification, a notification when a sterilization operation or a cleaning operation is completed, and a notification when a condition is abnormal. The indicator may be installed in the second body part 11b to guide the sterilization area.

In addition, the output unit 160 may include an led, an oled, an amoled display, or the like. Here, when the output unit 160 is provided as a touch screen, the output unit 160 may perform a function of the input unit 170. In this case, a separate input unit 170 may not be provided according to selection, and an input unit 170 that performs a limited function such as volume control, power button, and home button may be provided.

The input unit 170 may obtain a signal corresponding to the user's input. For example, the input unit 170 may obtain the user's input for performing the sterilization operation or the cleaning operation. For example, the input unit 170 may obtain the user's input related to the power on-off, the mode, intensity, time, and pattern of the sterilization operation or the cleaning operation.

In addition, the input unit 170 may include a keyboard, a keypad, a button, a jog shuttle, and a wheel. In addition, the user's input in the input unit 170 may be, for example, pressing, touching, and dragging the button. In addition, when the output unit 160 is implemented as a touch screen, the output unit 160 may perform the role of the input unit 170.

According to an embodiment, the input unit 170 may be configured as a separate module wirelessly or wiredly connected to the IPL sterilization device 100. For example, the IPL sterilization device 100 may obtain the input for sterilization or cleaning from the user through the input unit 170 configured as a separate module given to the user's hand. For example, the IPL sterilization device 100 may obtain the input for sterilization or cleaning from the mobile terminal, and in this case, the IPL sterilization device 100 may include a separate communication unit (not shown). Here, the mobile terminal may include a desktop PC, a mobile phone, a smart phone, a laptop computer, a personal digital assistant (PDA), a portable multimedia player (PMP), a slate PC, a tablet PC, an ultrabook, a wearable device, and the like.

The controller 180 may control each configuration of the IPL sterilization device 100 or process and calculate various information. For example, the controller 180 may control capacitor 120, lamp 130, and the circuit unit 140 to output pulse light to sterilize the sterilized organism.

The controller 180 may be implemented in software, hardware, and a combination thereof. For example, the controller 130 may be implemented in hardware a field programmable gate array (FPGA), an application specific integrated circuit (ASIC), a semiconductor chip, and other various electronic circuits. For example, the controller 180 may be implement as a logic program executed in accordance with the hardware described above, or in various computer languages, etc. In addition, the controller 180 may be of any type including, but not limited to, a microprocessor, a microcontroller, a digital signal processing device (dsp), or any combination thereof.

In the following description, if there is no separate reference, it can be understood that the operation of the IPL sterilization device 100 is performed under the control of the controller 180.

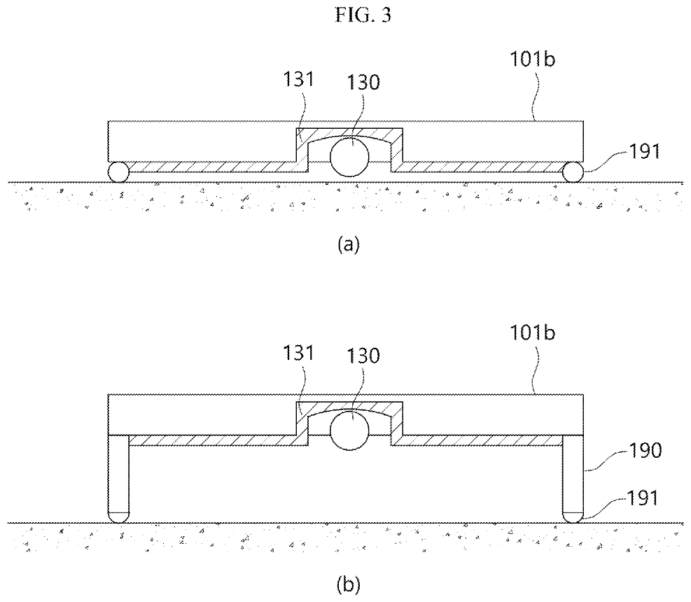

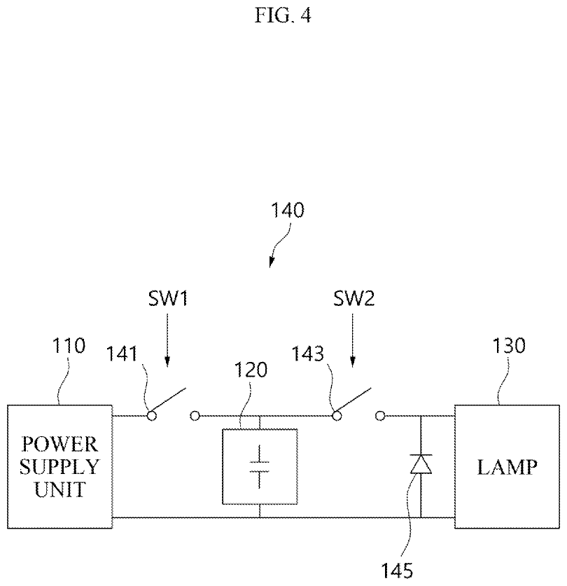

FIG. 4 is a circuit diagram illustrating the circuit unit 140 of the IPL sterilization device according to the first embodiment.

The circuit unit 140 may be a circuit for performing trigger driving. The circuit unit 140 may be connected to the power supply unit 110 to output a trigger voltage to the lamp 130.

The circuit unit 140 may be electrically connected to the power supply unit 110, the capacitor 120, and the lamp 130.

The circuit unit 140 may include a first switch 141, a second switch 143, and a diode 145.

The first switch 141 may be connected between the power supply unit 110 and the capacitor 120. The first switch 141 may connect the power supply unit 110 and the capacitor 120 in parallel.

The second switch 143 may be connected between the capacitor 120 and the lamp 130. The second switch 143 may connect the capacitor 120 and the lamp 130 in parallel.

The diode 145 may be connected between the second switch 143 and the lamp 130. The diode 145 may be connected in parallel with the lamp 130. The diode 145 is connected in parallel with the lamp 130 to prevent voltage or current flowing in the reverse direction to the lamp 130 to protect the lamp 130.

The first switch 141 and the second switch 143 may be controlled by a switching signal. The first switch 141 and the second switch 143 may be controlled by a control signal output from the controller 180.

The first switch 141 may be controlled by a first switching signal (SW1), and the second switch 143 may be controlled by a second switching signal (SW2). The first switching signal (SW1) and the second switching signal (SW2) may be output from the controller 180.

The first switch 141 may electrically connect one end of the power supply unit 110 and one end of the capacitor 120 when the first switching signal (SW1) is high. The first switch 141 may be opened so that when the first switching signal (SW1) is at a low level, one end of the power supply unit 110 and one end of the capacitor 120 are not electrically connected.

The second switch 143 may electrically connect one end of the capacitor 120 and one end of the lamp 130 when the first switching signal (SW1) is high. The second switch 143 may be opened so that when the second switching signal (SW2) is at a low level, one end of the capacitor 120 and one end of the lamp 130 are not electrically connected.

Since the second switch 143 may output a pulse to the lamp 130 by opening and shorting, it may serve as a pulse driving unit. When the lamp 130 is a xenon lamp, the xenon lamp may output pulsed light by the pulse.

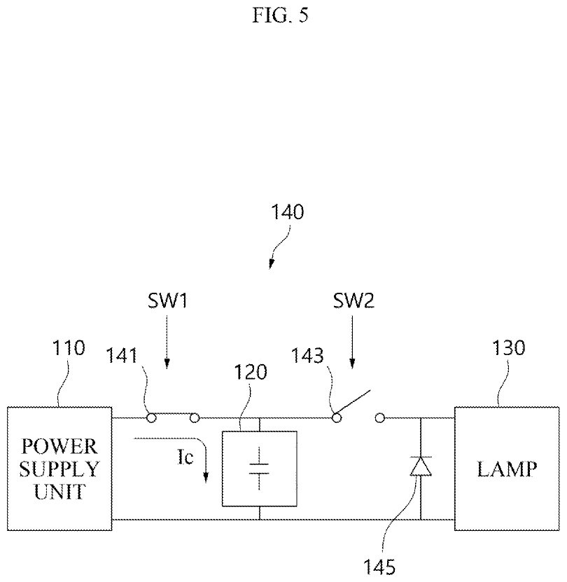

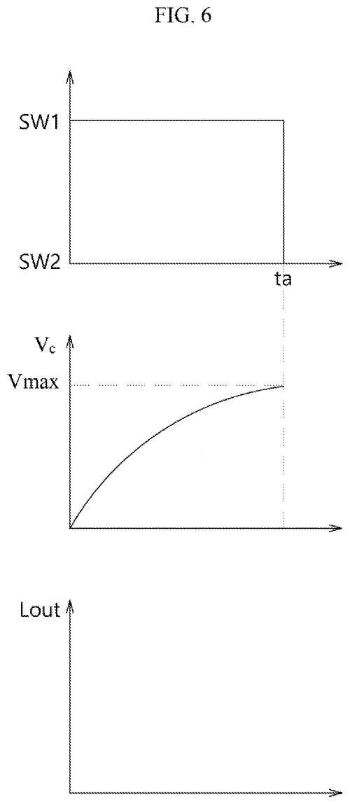

FIG. 5 is a circuit diagram illustrating when the circuit unit of the IPL sterilization device is in a charging state according to the first embodiment. FIG. 6 is a diagram illustrating the capacitor voltage and the light output from the lamp when the IPL sterilization device is in a charging state according to the first embodiment.

Referring to FIGS. 5 and 6, when the circuit unit 140 according to the first embodiment is in a charging state, the power supply unit 110 and the capacitor 120 may be connected.

The controller 180 may apply the first switching signal (SW1) at a high level and may apply the second switching signal (SW2) at a low level.

The first switch 141 may be shorted based on the first switching signal (SW1) of a high level, and the second switch 143 may be opened based on the second switching signal (SW2) of a low level.

The power supply unit 110 is electrically connected to the capacitor 120 by the shorted first switch 141, and the charging current (Ic) from the power supply unit 110 flows to the capacitor 120 to charge the capacitor 120. The capacitor 120 may be charged by the charging current (Ic). The capacitor 120 may be charged while having an RC delay and may be charged to a maximum charging voltage (Vmax). In this case, the second switch 143 is opened, and a voltage is not applied to the lamp 130, so that the state in which the lamp 130 does not emit light is maintained.

The controller 180 may detect the charge of the capacitor 120. The controller 180 may detect the amount of charge of the capacitor 120 by directly measuring the voltage of the capacitor 120, may detect the amount of charge of the capacitor 120 by detecting the charging current (Ic) flowing from the power supply unit 110 to the capacitor 120, or may detect the amount of charge of the capacitor 120 by monitoring the current or voltage output from the power supply unit 110. In the case where the power supply unit 110 is a battery, the controller 180 may detect the charge of the capacitor 120 by sensing the remaining amount or the change in the remaining amount of the battery.

When the controller 180 detects that the capacitor 120 has been charged to the maximum charging voltage (Vmax), the controller 180 may control the charging current (Ic) to prevent the flow of the capacitor 120 any longer. When the controller 180 detects that the capacitor 120 has been charged to the maximum charging voltage (Vmax), the controller 180 may open the first switch 141 to control the capacitor 120 not to be charged any more.

The controller 180 may control the charging voltage of the capacitor 120 to have a certain range. The controller 180 may control the first switching signal (SW1) so that the charging voltage of the capacitor 120 has a certain range. The controller 180 may control the charging amount of the capacitor 120 by changing the first switching signal (SW1) to a low level when the capacitor 120 has the maximum charging amount, and changing the first switching signal (SW1) to a high level again when the charging amount of the capacitor 120 is less than a certain value. In this case, the constant value may be a voltage when the lamp 130 is the minimum amount of charge for steady operation.

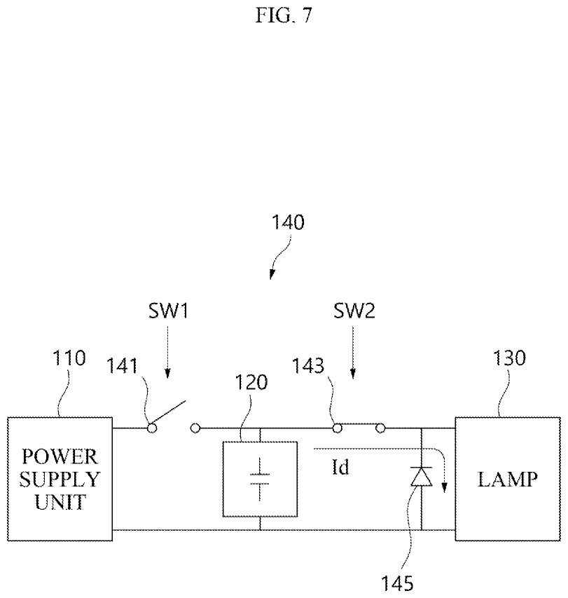

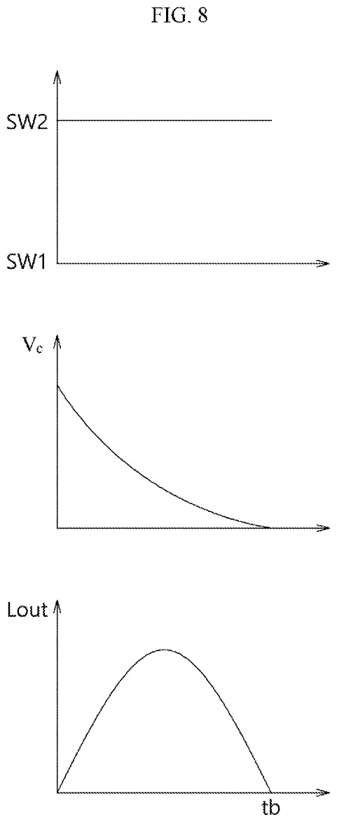

FIG. 7 is a circuit diagram illustrating when the circuit unit of the IPL sterilization device is in a light-emitting state according to the first embodiment. FIG. 8 is a diagram illustrating a capacitor voltage and light output from a lamp when the IPL sterilization device is in a light-emitting state according to the first embodiment.

Referring to FIGS. 7 and 8, when the circuit unit 140 according to the first embodiment is in a light-emitting state, the capacitor 120 and the lamp 130 may be connected.

The controller 180 may apply the second switching signal (SW2) at a high level and may apply the first switching signal (SW1) at a low level.

The second switch 143 may be shorted based on the second switching signal (SW2) of the high level, and the first switch 141 may be opened based on the first switching signal (SW1) of the low level.

The capacitor 120 may be connected to the lamp 130 by the second switch 143 that is shorted, and the lamp 130 may operate by flowing a driving current (Id) from the capacitor 120 to the lamp 130.

The high voltage (e.g., about 10,000 to 20,000 volts) charged to the capacitor 120 may be applied between the anode and cathode of the lamp 130, so that the gas in the tube of the lamp 130 may be ionized in the plasma state. The ionized gas may form a discharge passage rapidly for high voltage to make the lamp 130 an illuminated state.

The lamp 130 may output light having a maximum amount of light. The maximum amount of light at this time may be the amount of light that can sterilize the object.

The lamp 130 may emit light until the charge of the capacitor 120 is lost. The lamp 130 may emit light until the charge of the capacitor 120 is zero. Here, the case where the amount of charge becomes 0 has been described as an example, but the end time of the light emission of the lamp 130 may include the case where the amount of charge is not 0 but the amount of charge is a certain value or less.

The controller 180 may detect the charge of the capacitor 120. The controller 180 may detect the charge of the capacitor 120 by directly measuring the voltage of the capacitor 120, may detect the charge of the capacitor 120 by detecting the driving current (Id) flowing from the capacitor 120 to the lamp 130, or may detect the charge of the capacitor 120 by measuring the voltage of the lamp 130.

The controller 180 may control the driving current (Id) to prevent the flow to the lamp 130 when the controller 180 detects that the capacitor 120 has been discharged to the minimum voltage. The controller 180 may open the second switch 143 to control the capacitor 120 not to be discharged any more when the controller 180 detects that the capacitor 120 has been discharged to the minimum voltage.

The controller 180 may control the capacitor 120 to charge the capacitor 120 when the capacitor 120 is discharged. The controller 180 may control the first switching signal (SW1) and the second switching signal (SW2) so that the capacitor 120 may be recharged.

That is, the controller 180 may change the circuit unit 140 to a charging state when the capacitor 120 is discharged.

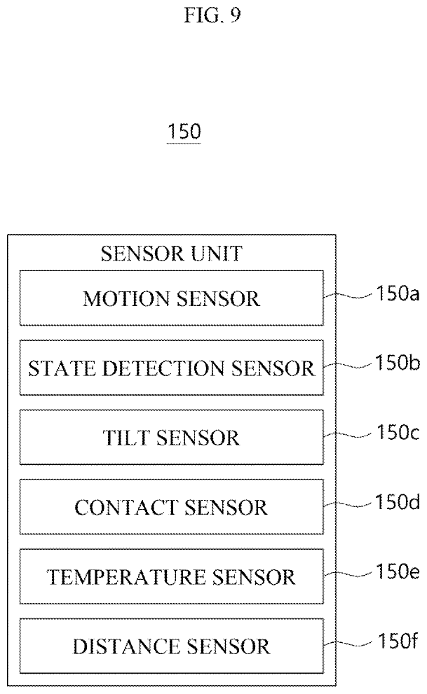

FIG. 9 is a diagram illustrating a sensor unit according to the first embodiment.

Referring to FIG. 9, the sensor unit 150 according to the first embodiment may include various sensors for sensing the surrounding environment of the IPL sterilization device 100 or the state of the IPL sterilization device 100.

For example, the sensor unit 150 may include a motion sensor 150a for sensing a motion of the IPL sterilization device 100. For example, the motion sensor 150a may be implemented as an acceleration sensor, a gyro sensor, a geomagnetic sensor, or the like disposed inside or outside the IPL sterilization device 100. The IPL sterilization device 100 may be controlled based on a sensing value obtained through the motion sensor 150a. Alternatively, the IPL sterilization device 100 may further include a wheel 191, and the sensor unit 150 may measure the number of rotations of the wheel 191. The controller 180 may calculate the movement (e.g., movement speed, rotation, etc.) of the IPL sterilization device 100 based on the number of rotations of the wheel 191 measured through the sensor 150. Specifically, when the wheels 191 of the IPL sterilization device 100 are arranged in pairs on the left and right sides, the controller 180 may determine the rotation based on the number of rotations of the left and right wheels 191 measured through the sensor 150. For example, when the number of rotations of the left and right wheels 191 of the IPL sterilization device 100 is the same, the controller 180 may determine that the right motion is performed, and when the number of rotations of the left and right wheels 191 of the IPL sterilization device 100 is different, the controller 180 may determine that the right motion is performed.

As another example, the sensor unit 150 may include a state detection sensor 150b for detecting the state of the object to be sterilized. For example, the state detection sensor 150b may be implemented as an illuminance sensor for detecting the illuminance of the organism to be sterilized, a temperature sensor for detecting the temperature of the organism to be sterilized, or the like. The IPL sterilization device 100 may be controlled based on a sensing value obtained through the state detection sensor 140b. When the state detection sensor 150b is an illuminance sensor, it may detect the color or brightness of the sterilized body.

As another example, the sensor unit 150 may include a tilt sensor 150c that detects when the body 101 is inverted or inclined. For example, the IPL sterilization device 100 may be controlled based on a sensing value obtained through the tilt sensor 150c.

As another example, the sensor unit 150 may include a contact sensor 150d that detects when a portion of the IPL sterilization device 100 has poor contact with the subject. For example, the IPL sterilization device 100 may be controlled based on a sensing value obtained through the contact sensor 150d.

As another example, the sensor unit 150 may include a temperature sensor 150e for sensing the temperature of the IPL sterilization device 100. For example, the temperature sensor 150e may be disposed inside or outside the body 101 and used to control the IPL sterilization device 100 so that the temperature of the IPL sterilization device 100 is not too high. That is, the IPL sterilization device 100 may be changed from an operation state to a standby state to be described later when a specific temperature or more is detected by the temperature sensor 150e.

As another example, the sensor unit 150 may include a distance sensor 150f for detecting a distance between a portion of the IPL sterilization device 100 and a subject to be sterilized. For example, the distance sensor 150f may be disposed inside or outside to detect the distance between the lamp 130 and the object to be sterilized. The IPL sterilization device 100 may be controlled based on a sensing value obtained through the distance sensor 150f. The distance sensor 150f may be installed on at least one surface of the housing in contact with the object to measure the distance to the object in contact with the IPL sterilization device 100.

The sensor unit 150 may output a sensor signal indicating a voltage reflecting a sensing value. Alternatively, the sensor unit 150 may compare the sensing value with a preset threshold value and output a sensor signal when the sensing value exceeds the preset threshold value.

Of course, the sensor unit 150 may be implemented differently, including a pressure sensor, and the like, and is not limited to the above-described description.

In addition, the controller 180 may not use the sensing value of the sensor unit 150 when it is autonomously confirmed regarding information detected by the sensor unit 150.

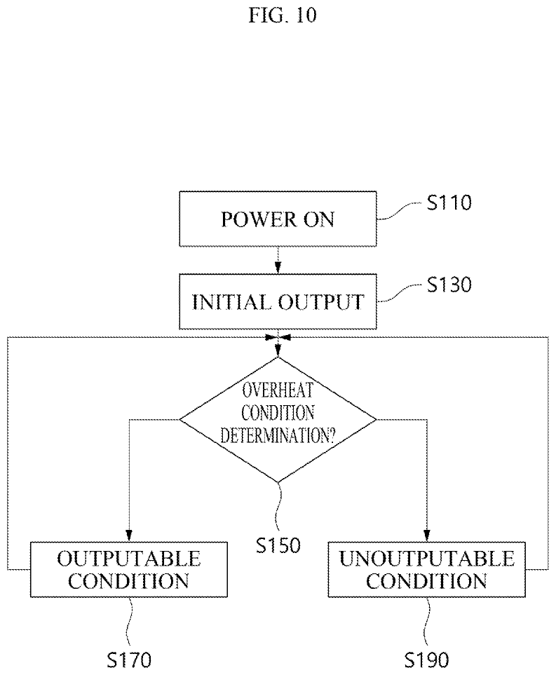

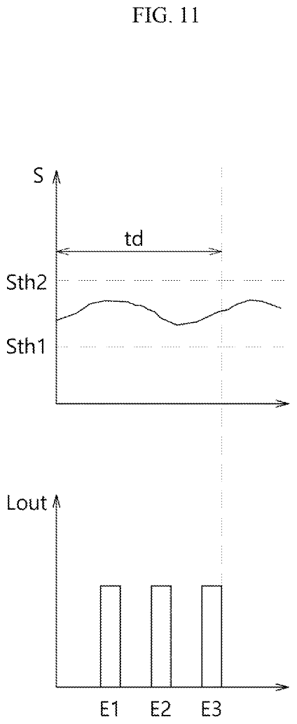

FIG. 10 is a flowchart illustrating the operation of the IPL sterilization device according to the first embodiment, and FIG. 11 is a waveform diagram related to the operation of the IPL sterilization device according to the first embodiment.

Referring to FIG. 10, the IPL sterilization device according to the first embodiment may include a power ON step (S110), an initial output step (S130), an object overheat condition determination step (S150), an outputable condition setting step (S170), and an unoutputable condition setting step (S190).

The power of the IPL sterilization device may be turned on (S110).

The IPL sterilization device 100 may be powered on by the user's control. The IPL sterilization device 100 may be powered on by input through the user's input unit 170.

When the power of the IPL sterilization device 100 is turned on, the IPL sterilization device may operate in an initial output step (s130).

In the initial output step, the IPL sterilization device 100 may operate so that the lamp 130 outputs light under the control of the controller 180.

Because the IPL sterilization device 100 is not likely to be an object overheat condition in the initial output state immediately after the power of the IPL sterilization device 100 is ON, the lamp 130 may be controlled to output light without determining the object overheat condition in the initial output step.

Even in this case, the IPL sterilization device 100 may measure the distance (d) of the object and control the IPL sterilization device 100 according to the distance to the object.

When the initial output step is finished, the IPL sterilization device 100 may determine whether the object is in an object overheat condition (S150).

The step of determining the object overheat condition may be a step in which the controller 180 of the IPL sterilization device 100 determines whether the object is in an object overheat condition using the sensor 150. The controller 180 may determine whether the object is in an overheat condition based on the sensing value measured by the sensor unit 150.

The controller 180 may determine that the sterilization object is in an overheat condition when the temperature of the object is equal to or higher than a predetermined temperature through the temperature measurement of the object. The controller 180 may measure the temperature of the object through the state sensing sensor 150b, and if the measured result is equal to or greater than a predefined value, it may determine that the object is in an overheat condition.

The controller 180 may determine that the object is overheating when the IPL sterilization device 100 is stopped or repeats the light output without a big motion. The controller 180 may determine an object overheat condition based on a sensing value output from at least one of the motion sensor 150a, the tilt sensor 150c, the contact sensor 150d, or the distance sensor 150f.

The controller 180 may determine that the object is overheating if light output is performed more than a predetermined number of times in a state when there is no change in the sensing value measured by the motion sensor 150a. The controller 180 may determine that the object is overheating when light output is performed more than a predetermined number of times while the sensing value measured by the motion sensor 150a is within a predetermined range.

The controller 180 may determine that the object is overheating if light output is performed more than a predetermined number of times in a state when there is no change in the sensing value measured by the contact sensor 150d. In addition, the controller 180 may determine that the object is overheating if the IPL sterilization device 100 operates for more than a predetermined time in a state when the IPL sterilization device 100 is determined to maintain contact with the object by the contact sensor 150d.

The controller 180 may determine that the object is overheating if light output is performed more than a predetermined number of times in a state when there is no change in the sensing value measured by the distance sensor 150f. The controller 180 may determine that the object is overheating when the sensing value measured by the distance sensor 150f is within a predetermined range and light output is performed more than a predetermined number of times.

The controller 180 may determine that the object is overheating when the sensing value has elapsed more than a predetermined time (td) while maintaining a value between the first sensing threshold value (Sth1) and the second sensing threshold value (Sth2).

Alternatively, the controller 180 may determine that the object is overheating when the output of light pulses more than a predetermined number of times is performed while the sensing value maintains a value between the first sensing threshold value (Sth1) and the second sensing threshold value (Sth2).

For example, when the sensing value maintains a value between the first sensing threshold value (Sth1) and the second sensing threshold value (Sth2) while the first light emission (E1), the second light emission (E2), and the third light emission (E3) are performed, the object may be determined that the object is overheating.

The controller 180 may change the IPL sterilization device 100 to an outputable condition if the object is not in the overheat state after the object overheat condition is determined (S170).

In the outputable condition, the controller 180 may perform a sterilization operation of the IPL sterilization device 100.

The IPL sterilization device 100 may repeat the light emission state and the charging state during the operation. The IPL sterilization device 100 may control the lamp 130 to emit light through the connection as shown in FIG. 7 when it is in the light emission state, and may control the capacitor 120 to be charged through the connection as shown in FIG. 5 when it is in the charging state.

In the IPL sterilization device 100 according to the first embodiment, the controller 180 may alternately output the first switching signal (SW1) and the second switching signal (SW2). The controller 180 may control the first switch 141 and the second switch 143 to be alternately opened/shorted. The controller 180 may repeatedly change the charging state when the light emission state is terminated, and change the light emission state when the charging state is terminated. When it is an operational state, the controller 180 may control the lamp 130 to repeat the output a plurality of times. The operational state is maintained only when the IPL sterilization device 100 is within a safe distance, and the operational state is terminated when the distance between the IPL sterilization device 100 and the object is not a safe distance.

Alternatively, the operational state may be maintained only when the IPL sterilization device 100 is not in the overheat condition, and the operational state may be terminated when the IPL sterilization device 100 is in the overheat condition.

The operational state may be started by a user input. That is, even when the IPL sterilization device 100 is within a safe distance, the operational state may be maintained in the standby state, and when there is a user input, the operational state may be changed to the operational state, and the second switching signal (SW2) may be initially output to a high level.

The operational state may be terminated by a user input. That is, even when the operational state is started by the user input, the IPL sterilization device 100 may be changed to the operational state when a termination command is input by the user input.

Even when the operational state is started by the user input, the sensing value is maintained in the state in which light emission is performed within a predetermined time or a predetermined number of times, it may be determined as the object overheated state.

Although the initial output and the outputable state are separately illustrated and described, the operation of the IPL sterilization device 100 in the initial output and the outputable state may be the same. That is, the IPL sterilization device 100 may repeatedly determine whether it corresponds to the object overheat condition or in the outputable state, and perform a function according to the result of the determination.

The controller 180 may change the IPL sterilization device 100 to the unoutputable state when the object is in the overheat state after the object overheat condition is determined (S190).

In the unoutputable state, the controller 180 may prevent light from the lamp 130 to be output and control the capacitor 120 to be charged.

The controller 180 may control the capacitor 120 to charge as shown in FIG. 5 in an unoutputable state.

The controller 180 may output the first switching signal (SW1) at a high level to make the first switch 141 short state, and output the second switching signal (SW2) at a low level to make the second switch 143 open state. Thus, the charging current (Ic) is supplied from the power supply 110 to the capacitor 120.

When the charging voltage Vc of the capacitor 120 reaches the maximum charging voltage, the controller 180 may output the first switching signal (SW1) at a low level and may open the first switch 141. Thereafter, when the voltage of the capacitor 120 may be changed to a certain value or less due to natural discharge, the first switching signal (SW1) may be output again at a high level and the capacitor 120 may be charged again. This process is defined as a maintenance step, and the controller 180 controls the charging voltage of the capacitor 120 to be maintained within a certain range. However, this maintenance step may be omitted, and when the maintenance step is omitted, the controller 180 may output the first switching signal (SW1) at a high level in the standby state to make the first switch 141 short state, and may control the voltage of the power supply 110 to be continuously applied to the capacitor 120 during the standby state.

The controller 180 may change the lamp to the outputable state when the object leaves the object overheat condition after the object reaches the object overheat condition and is changed to the unoutputable state. When a predetermined time elapses from the change to the unoutputable state, the controller 180 may determine that the object overheat condition has left, and change the lamp to the outputable state.

In addition, the controller 180 may continuously measure the temperature of the object in the unoutputable state, and when the temperature of the object is measured as the temperature that leaves the unoutputable state, determine that the object overheat condition has left, and change the lamp to the outputable state.

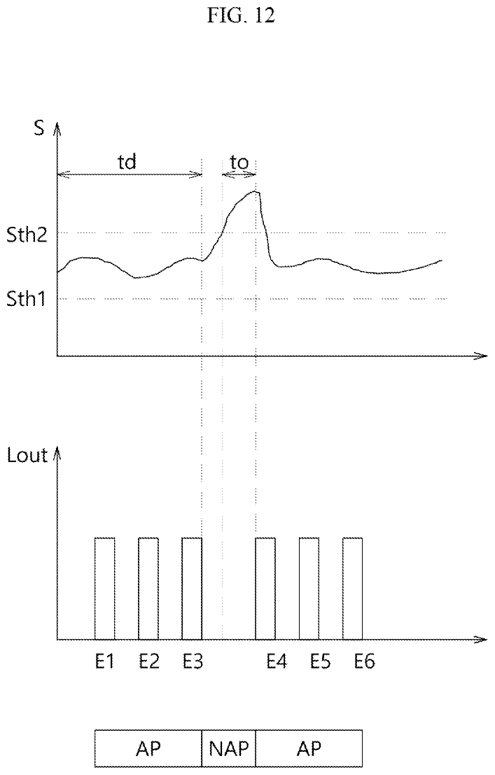

FIG. 12 is a diagram illustrating the object overheat condition leaving in the unoutputable state of the IPL sterilization device according to the first embodiment.

Referring to FIG. 12, the IPL sterilization device according to the first embodiment performs the first light emission E1, the second light emission E2, and the third light emission E3 in the outputable state (AP).

In the state in which the first light emission E1, the second light emission E2, and the third light emission E3 are performed, the sensing value is measured as a value between the first sensing threshold value Sth1 and the second sensing threshold value Sth2, and thus the controller 180 determines that the IPL sterilization device 100 is the overheat condition, and changes the IPL sterilization device 100 to the unoutputable state NAP.

At this time, the controller 180 may determine that the threshold time elapses using the sensing value holding time td of the outputable state AP as the overheat condition.

The controller 180 obtains the sensing value from the sensor even in the unoutputable state (NAP). Even if the sensing value leaves a stop period between the first sensing threshold value (Sth1) and the second sensing threshold value (Sth2), the controller 180 may determine the object overheat condition leaving of the IPL sterilization device based on the leaving time (to).

The controller 180 may terminate the unoutputable state (NAP) and change the IPL sterilization device 100 to the outputable state AP when the leaving time after the object overheat condition leaving is a predetermined time.

That is, even if the sensing value is measured by a value equal to or greater than the second sensing threshold value (Sth2), the controller 180 may change the IPL sterilization device 100 to the outputable condition (AP) after a certain time has elapsed while maintaining the value equal to or greater than the second sensing threshold value (Sth2).

As a result, even when the IPL sterilization device 100 has a temporary motion, it may be determined that the object overheat condition of the object is maintained, and the object damage may be more effectively prevented.

The controller 180 may change the IPL sterilization device 100 to an output enable state (AP) when the separation time (to) is equal to or more than a predetermined time, and control the fourth light emission (E4), the fifth light emission (E5), and the sixth light emission (E6) to be performed. In addition, when the sensing value maintains the stop period while the sixth light emission (E6) is performed, the IPL sterilization device may be changed to an unoutputable state (nap).

Although it was described in the drawing that the object overheat condition is determined after performing three times of light emission, several times of light emission may be performed by the number of times exceeding three times of light emission if a certain time elapses while leaving the stop period during the light emission process. In other words, if the IPL sterilization device 100 is not stationary, the sensing value will operate with the stationary period away, and in this case, the IPL sterilization device 100 may perform a plurality of times within the capacity range.

Here, the first sensing threshold value (Sth1), the second sensing threshold value (Sth2) and the stop period based on this may be an absolute value or a relative value. When the sensing value for determining the object overheat condition is the motion sensor 150a, the contact sensor 150d, and the distance sensor 150f, the first sensing threshold Sth1 and the second sensing threshold Sth2 may be absolute values. In other words, in this case, since the sensing values are values directly indicating the movement of the IPL sterilization device 100, it is appropriate to determine whether the object is in a situation in which it may overheat by taking the standard as an absolute value.

Even when the sensing value for determining the object overheat condition is the temperature sensor 150e, the first sensing threshold value Sth1 and the second sensing threshold value Sth2 may be absolute values. In this case, it is appropriate to measure the internal temperature of the IPL sterilization device 100 and change it to an unpowered state when it is overheated.

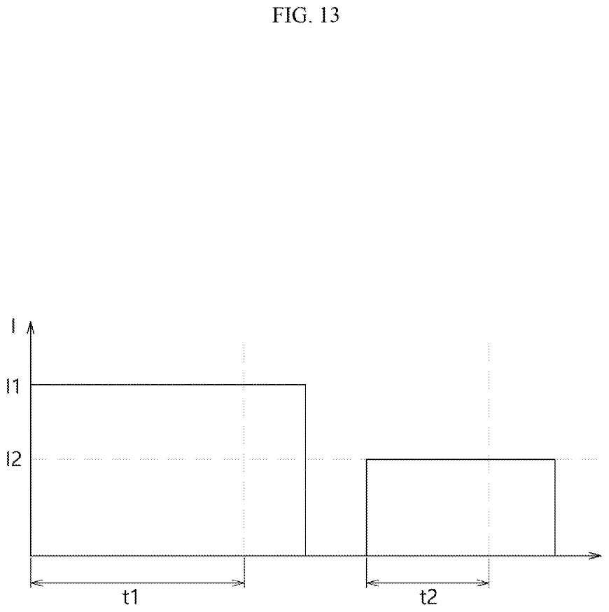

When the sensing value for determining the object overheat condition is the state sensing sensor 150b, the first sensing threshold value (Sth1) and the second sensing threshold value (Sth2) may be absolute or relative. When the sensing value indicates the illuminance of the object, the first sensing threshold value (Sth1) and the second sensing threshold value (Sth2) may be relative values. If the sensing value indicates the illuminance of the object, the reference value measured according to the color of the object will be different, and in this case, the first sensing threshold value (Sth1) and the second sensing threshold value (Sth2) may be set based on when the first light emission operation is performed. Alternatively, when a sensing value is input, the controller 180 may update the sensing value in real time, determine whether the updated sensing value is within a stop period, and determine an object overheat condition. The object overheat condition determination by an illuminance sensor will be described again in FIG. 13.

In order to determine the overheat condition, when the temperature of the object is directly measured, the object may be determined to be overheated when the temperature is outside of a predetermined range and may be changed to a non-output state.

FIG. 13 is a diagram for explaining the object overheat condition determination according to an illuminance sensor in an IPL sterilization device according to the first embodiment.

Referring to FIG. 13, the IPL sterilization device according to the first embodiment may determine an object overheat condition of the object by sensing values measured by the state sensing sensor 150b. The IPL sterilization device 100 may determine an object overheat condition of the object based on an illuminance value measured when the state detection sensor 150b is an illuminance sensor.

When the measured illuminance value has the first illuminance value (I1), the IPL sterilization device 100 may determine that the object is overheated when the first time (t1) elapses while the illuminance value maintains the first illuminance value (I1).

When the measured illuminance value has a second illuminance value (I2), the IPL sterilization device 100 may determine that the object has overheated when the second time (t2) elapses while the illuminance value maintains the second illuminance value (I2).

The first illuminance value (I1) may be a value greater than the second illuminance value (I2), and the first time (t1) may be a time longer than the second time (t2).

When the energy applied to the object by the lamp 130 is the same, the energy absorbed when the illuminance of the object is large may be relatively small, and when the illuminance of the object is small, the energy absorbed may be relatively large. That is, when the object is close to white, the energy absorbed by the light emission of the lamp 130 may be relatively small, and when the object is close to black, the energy absorbed by the light emission of the lamp 130 may be relatively large.

Therefore, when the illuminance value measured by the object is relatively large, the object overheat condition may be set to a long time. In addition, when the illuminance value measured by the object is relatively small, the object overheat condition may be set to a relatively short period.

As a result, it is possible to effectively sterilize and prevent the object from being damaged by determining the object overheat condition differently due to the color or brightness of the object.

Here, the illuminance value measured by the illuminance sensor may be a value measured during a period during which the lamp 130 emits light by the IPL apparatus 100. That is, the light output by the lamp 130 may be measured as an illuminance value reflected by the object. Since the lamp 130 is output in the form of pulsed light, it is possible to determine overheating of the object by using the accumulated illuminance value of the period corresponding to the pulse period. As a result, the illuminance value may reflect the actual absorption rate of the object with respect to the light output from the lamp 130.

Alternatively, the illuminance value measured by the illuminance sensor may be a value measured in addition to the period during which the lamp 130 emits light by the IPL apparatus 100. The light output by the lamp 130 is pulsed light, and because the pulse period may be relatively short, the period in which the lamp 130 emits light may be excluded and the illuminance value may be measured. In this case, the overheating of the object may be determined by using the value deleted for the period during which the lamp 130 emits light after accumulating the illuminance value.

In this case, the IPL apparatus 100 may further include a light shielding structure to block light output from the lamp 130 from being incident on the illuminance sensor. By the light shielding structure, light output from the lamp 130 may be prevented from being directly incident on the illuminance sensor, thereby preventing the illuminance sensor from being damaged, and implementing the illuminance sensor with a sensor that detects a low amount of light.

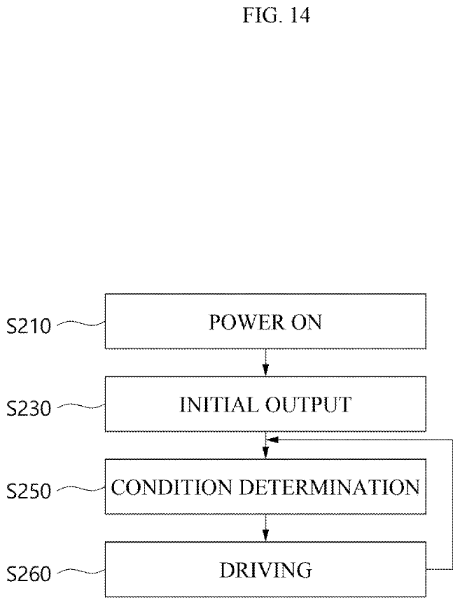

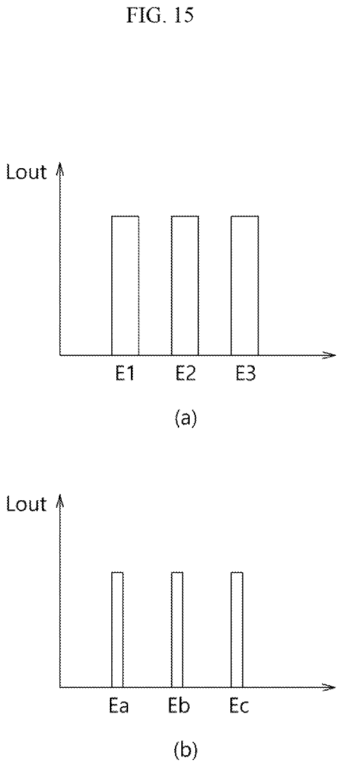

FIG. 14 is a flowchart illustrating the driving of the IPL sterilization device according to the second embodiment, and FIG. 15 is a waveform diagram illustrating the standby condition driving according to the second embodiment.

The IPL sterilization device according to the second embodiment is the same as the first embodiment, except that standby conditions are added to the object overheat condition determination and standby conditions are added in the driving step compared to the first embodiment. Therefore, in describing the second embodiment, the same drawing numbers are given to a configuration common to the first embodiment and detailed descriptions are omitted.

Referring to FIGS. 14 and 15, the IPL sterilization device 100 according to the second embodiment may include a power on step (s210), an initial output step (s230), a condition determination step (s250), and a driving step (s260) according to conditions.

The power of the IPL sterilization device 100 may be turned on (s210).

When the power of the IPL sterilization device 100 is turned on, the IPL sterilization device may operate in an initial output step (s130).

When the initial output step is finished, the IPL sterilization device 100 may determine whether the object is in an object overheat condition (S250).

The controller 180 may determine whether the object corresponds to an overheat condition, not an overheat condition, and a standby condition.

The standby condition may be a condition of an intermediate step between the case where the object is overheated and the case where it is not overheated. In other words, the standby condition may be a condition that indicates a state in which the object is not overheated but is room for overheating. The standby condition may be a section set before and after the overheat condition. That is, the section before the object is determined to be an object overheat condition or the section immediately after the object deviates from the object overheat condition may be determined to be the standby condition. Alternatively, when the temperature of the object or the temperature of the IPL sterilization device 100 is above a certain temperature that is lower than the overheat condition temperature, it may be determined as a standby condition.

The controller 180 may operate the IPL sterilization device 100 based on condition determination (s260).

The controller 180 may stop output by the lamp 130 when the object is an overheat condition, and emit the lamp 130 when the object is not an overheat condition. In addition, in the case of standby condition, the lamp 130 may be controlled to output lower energy than in the case of normal operation.

For example, as shown in FIG. 15(a), when the controller 180 operates normally, if the first light emission (E1), the second light emission (E2), and the third light emission (E3) having the first pulse width are performed through the lamp 130, the standby condition driving may be performed by the lamp 130 as shown in FIG. 15(b), the ath light emission (Ea), the bth light emission (Eb), and the cth light emission (Ec) having the second pulse width. In this case, the second pulse width may be smaller than the first pulse width.

Through the standby state driving of the IPL sterilizer 100, it may be notified that the object is imminent to an overheat condition. Alternatively, the IPL sterilization device 100 may delay the time when the object corresponds to an object overheat condition through the standby state driving, thereby enabling more efficient sterilization.

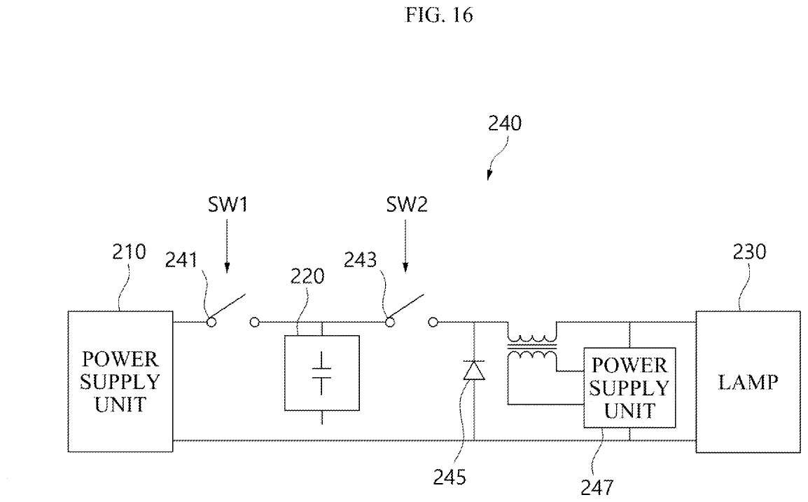

FIG. 16 is a diagram illustrating a circuit unit of an IPL sterilization device according to the third embodiment.

The circuit unit 240 may be a circuit for performing shimmer driving. The circuit unit 240 may be connected to the power supply unit 210 to output the shimmer voltage to the lamp 230.

The circuit unit 240 may be electrically connected to the power supply unit 210, the capacitor 220, and the lamp 230.

The circuit unit 240 may include a first switch 241, a second switch 243, a diode 245, and a shimmer driving unit 247.

The first switch 241 may be connected between the power supply unit 210 and the capacitor 220. The first switch 241 may connect the power supply unit 210 and the capacitor 220 in parallel.

The second switch 243 may be connected between the capacitor 220 and the shimmer driving unit 247. The second switch 243 may connect the capacitor 220 and the shimmer driving unit 247 in parallel.

The diode 245 may be connected between the second switch 243 and the shimmer driving unit 247. The diode 245 may be connected in parallel to the simmer driving unit 247. The diode 145 may be connected in parallel with the simmer driving unit 247 to prevent voltage or current flowing in the reverse direction to the simmer driving unit 247 and the lamp 230, thereby protecting the simmer driving unit 247 and the lamp 230.

The shimmer driving unit 247 may be connected between the second switch 243 and the lamp 230. The shimmer driving unit 247 may include a transformer. The shimmer driving unit 247 may supply voltage so that the lamp 230 may maintain a preliminary light emission state. The shimmer driving unit 247 may continuously apply a constant current to the lamp 230 to maintain the preliminary light emission state of the lamp 230. The preliminary light emission state may be a state having a small amount of light emission based on a smaller energy than the light emission operation that emits light by applying a high voltage to the lamp 230. The simmer driving unit 247 may provide a voltage that may maintain the ionized state of the gas in the tube of the lamp 130 in the plasma state. By maintaining the preliminary light emission state of the lamp 230 by the shimmer driving unit 247, it is possible to emit the lamp 230 with a small voltage compared to the first embodiment. In addition, by controlling the timing of the driving voltage, it is advantageous in that the amount of light emission of the lamp 230 may be controlled.

The first switch 241 and the second switch 243 may be controlled by a switching signal. The first switch 241 and the second switch 243 may be controlled by a control signal output from the controller 180.

The first switch 241 may be controlled by a first switching signal (SW1), and the second switch 243 may be controlled by a second switching signal (SW2). The first switching signal (SW1) and the second switching signal (SW2) may be output from the controller 180.

The first switch 241 may electrically connect one end of the power supply unit 210 and one end of the capacitor 220 when the first switching signal (SW1) is a high level. The first switch 241 may be opened so that one end of the power supply unit 210 and one end of the capacitor 220 are not electrically connected when the first switching signal (SW1) is a low level.

The second switch 243 may electrically connect one end of the capacitor 220 and one end of the shimmer driving unit 247 when the first switching signal (SW1) is a high level. The second switch 243 may be opened so that one end of the capacitor 220 and one end of the shimmer driving unit 247 are not electrically connected when the second switching signal (SW2) is a low level.

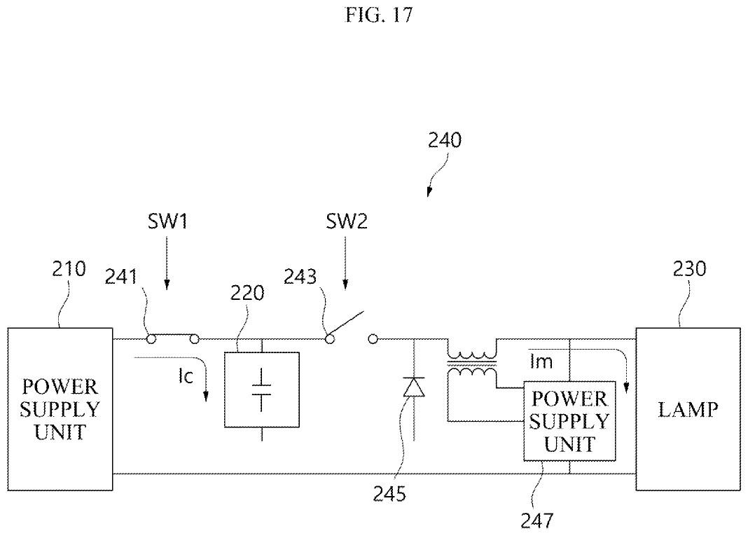

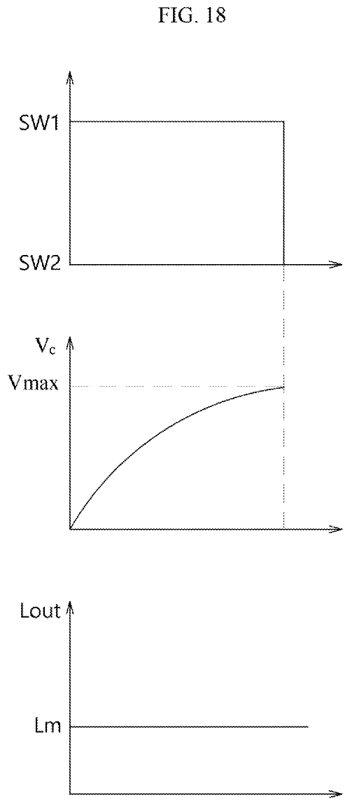

FIG. 17 is a circuit diagram illustrating a case where the circuit unit of the IPL sterilization device is in a charging state according to the third embodiment. FIG. 18 is a diagram illustrating a capacitor voltage and light output from a lamp when the IPL sterilization device is in a charging state according to the third embodiment.

Referring to FIGS. 17 and 18, the circuit unit 240 according to the third embodiment may be connected to the power supply unit 210 and the capacitor 220 when in a charging state.

The first switch 141 is shorted based on the first switching signal (SW1) of a high level, and the second switch 143 is opened based on the second switching signal (SW2) of a low level.

The power supply unit 210 may be electrically connected to the capacitor 220 by the shorted first switch 241, and the charging current (Ic) from the power supply unit 210 may flow to the capacitor 220 to charge the capacitor 220. The capacitor 220 may be charged by the charging current (Ic). The capacitor 220 may be charged with RC delay and may be charged to a maximum charging voltage Vmax. At this time, the second switch 243 is opened, so that the current of the capacitor 220 or the power supply unit 210 is not applied to the lamp 230.

The charging and charging maintenance of the capacitor 220 has the same characteristics as in the first embodiment.

The shimmer driving unit 247 maintains a state electrically connected to the lamp 230. The shimmer driving unit 247 may supply a maintenance current Im that may maintain the preliminary light emission state of the lamp 230. The lamp 230 may maintain the preliminary light emission state having Lm light amount by the shimmer driving unit 247 supplying the maintenance current (Im) to the lamp 230.

A separate voltage may be supplied to the shimmer driving unit 247. The separate voltage may be supplied from the power supply unit 210, but is not limited thereto.

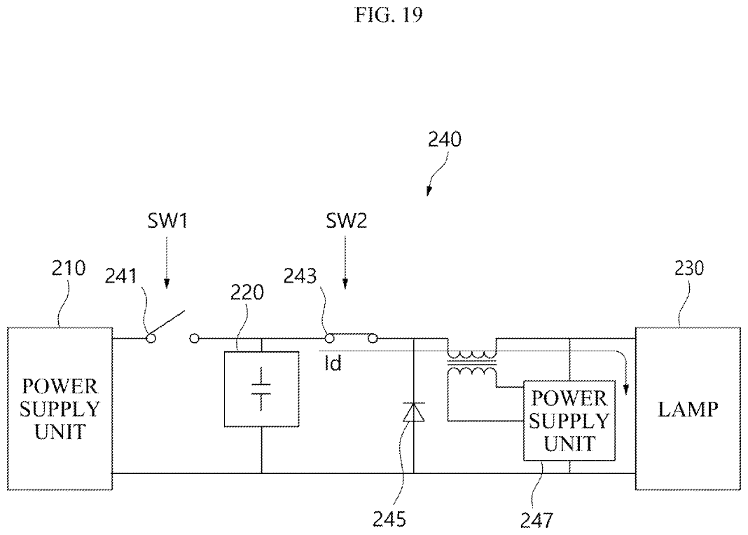

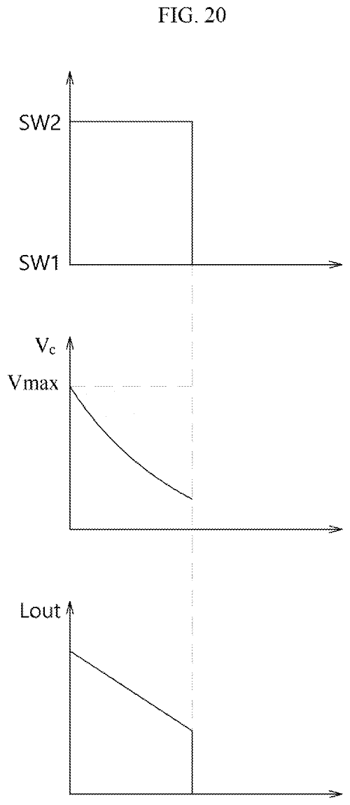

FIG. 19 is a circuit diagram illustrating a case where the circuit unit of the IPL sterilization device is in a light emission state according to the third embodiment. FIG. 20 is a diagram illustrating a capacitor voltage and light output from a lamp when the IPL sterilization device is in a light emission state according to the third embodiment.

Referring to FIGS. 19 and 20, when the circuit unit 240 is in a light emission state, the capacitor 220 may be connected to the shimmer driving unit 247 and the lamp 230.

The controller 180 applies a second switching signal (SW2) at a high level and applies

The second switch 243 may be short-circuited based on the second switching signal (SW2) of the high level, and the first switch 241 may be opened based on the first switching signal (SW1) of the low level.

The capacitor 220 may be connected to the lamp 230 and the shimmer driving unit 247 by the short-circuited second switch 243, and the driving current (Id) from the capacitor 220 flows to the lamp 230 to operate the lamp 230. At this time, the shimmer driving unit 247 may be electrically connected to the lamp 230, and the current of the shimmer driving unit 247 may flow to the lamp 230.

The high voltage (e.g., about 10,000 to 2,000,000 volts) charged to the capacitor 220 may be applied between the anode and the cathode of the lamp 230, so the gas in the tube of the lamp 230 may be additionally ionized in the plasma state. The ionized gas may form a discharge passage rapidly for the high voltage to bring the lamp 230 into an illuminated state. The illumination may have a larger light amount compared to the preliminary light emission.

The lamp 230 may output a light amount that may sterilize the object.

The lamp 230 may output a light amount controlled based on the second switching signal (SW2). The lamp 230 may emit light by the amount of light corresponding to the discharge amount of the capacitor 220 in the capacitor 120 during the period in which the second switch 243 is short-circuited. That is, the lamp 230 may emit light for a time corresponding to the amount transferred from the capacitor 220 to the lamp 230. In other words, the lamp 230 may maintain the light emission state by the time that the high level of the second switching signal (SW2) is maintained. When the second switching signal (SW2) is changed to the low level again, the lamp 230 may return to the preliminary light emission state again.

By controlling the light emission amount of the lamp 230 by the second switching signal (SW2), the IPL sterilization device according to the third embodiment is capable of controlling the light emission amount. Because the circuit configuration is capable of controlling the light emission amount according to the type of the object and the degree to be sterilized, power consumption may be reduced, and the design is capable of being more suitable for the operating condition.

The method according to the embodiment may be implemented in the form of program instructions that may be performed through various computer means and recorded in a computer-readable medium. The computer-readable medium may include program instructions, data files, data structures, etc., alone or in combination. The program instructions recorded in the medium may be specially designed and configured for the embodiment or those known to those skilled in the art of computer software. Examples of the computer-readable recording medium include magnetic media such as hard disks, floppy disks, and magnetic tape, optical media such as CD-ROM and DVD, magneto-optical media such as floptical disks, and hardware devices specially configured to store and perform program instructions such as ROM, RAM, flash memory, etc. Examples of the program instructions include higher language code that may be executed by a computer using an interpreter, as well as machine code created by a compiler. The hardware device may be configured to operate as one or more software modules to perform the operations of the embodiment, and vice versa.

Although the embodiments are described by the limited embodiments and drawings, those skilled in the art can have various modifications and modifications from the above descriptions. For example, appropriate results may be achieved even if the described techniques are performed in a different order from the described method, and/or if the elements of the described system, structure, apparatus, circuit, etc., are combined or combined in a different form from the described method, or replaced or substituted by other elements or equivalents.

Therefore, other implementations, other embodiments, and equivalents to the claims are also within the scope of the claims described below.

1. An IPL sterilization device comprising:
a lamp configured to output light including a visible light region to sterilize a region including a surface of an object;
a capacitor configured to transmit a voltage charged to the lamp;
and a controller configured to control the capacitor, wherein the controller is configured to:
control the lamp to be driven in an outputable state or an unoutputable state,
change the lamp to the unoutputable state when the object satisfies an overheat condition of the object in the outputable state, and
change the lamp to the outputable state when the object unsatifies the object overheat condition of the object after the lamp satisfies the object overheat condition of the object and changes the lamp to the unoutputable state,
wherein the outputable state is a state in which the lamp is capable of outputting light by applying a driving pulse to the lamp by the capacitor, and
wherein the unoutputable state is a state in which the lamp is not capable of outputting light because the capacitor does not output a driving voltage to the lamp.

2. The IPL sterilization device of claim 1, wherein the controller is configured to determine the object overheat condition when the temperature measured by a temperature sensor is higher than a predetermined temperature.

3. The IPL sterilization device of claim 1, wherein the controller is configured to determine the object overheat condition when the IPL sterilization device does not move during a predetermined period of time.

4. The IPL sterilization device of claim 1, wherein the controller is configured to determine the object overheat condition when a driving pulse is output a predetermined number of times while a sensing value output by one of a motion sensor, an illuminance sensor, or a contact sensor is within a certain range.

5. The IPL sterilization device of claim 1, wherein the controller is configured to determine the object overheat condition when a variation in a sensing value detected by one of a motion sensor, an illuminance sensor, or a touch sensor is within a predetermined range during a predetermined period of time.

6. The IPL sterilization device of claim 1, wherein the controller is configured to change the lamp the an outputable state when a predetermined period of time has elapsed while the object overheat condition is not satisfied after changing to the unoutputable state.

7. The IPL sterilization device of claim 1,
further comprising an illuminance sensor configured to measure the illuminance of the object,
wherein the controller is configured to:
determine the object overheat condition when the sensing value measured by the illuminance sensor is maintained for a first period of time with a first value, and
determine the object overheat condition of the object when the sensing value measured by the illuminance sensor is maintained for a second period of time with a second value,
wherein the first value is a larger value than the second value, and the first period is longer than the second period.

8. The IPL sterilization device of claim 7, wherein the illuminance sensor is configured to measure a color or an illuminance of the object in a section where the lamp emits.

9. The IPL sterilization device of claim 7, wherein the illuminance sensor is configured to measure a color or illuminance of the object in a section other than the section where the lamp emits.

10. The IPL sterilization device of claim 1, wherein the controller is configured to control the lamp to be driven in a standby state when the standby condition,
wherein the standby condition is a condition that has a temperature lower than a temperature of the object overheat condition.

11. The IPL sterilization device of claim 10, wherein the controller is configured to control a pulse having a different pulse width from the driving pulse to be applied to the lamp from the capacitor when the lamp is in the standby condition.

12. The IPL sterilization device of claim 11, wherein the pulse width applied to the lamp in the standby condition is smaller than the driving pulse applied to the lamp in the outputable state.

13. The IPL sterilization device of claim 7, further comprising a light shielding structure for blocking light output from the lamp from being incident on the illuminance sensor.
